(19) United States
(12) Patent Application Publication
(10) Pub. No.: US 2013/0096405 A1
(43) Pub. Date: Apr. 18, 2013

(54) FINGERTIP PULSE OXIMETER

(71) Applicant: Alejandro Garfio, West Covina, CA (US)

(72) Inventor: Alejandro Garfio, West Covina, CA (US)

(73) Assignee: MASIMO CORPORATION, Irvine, CA (US)

(21) Appl. No.: 13/571,910

(22) Filed: Aug. 10, 2012

Related U.S. Application Data
(60) Provisional application No. 61/523,161, filed on Aug. 12, 2011.

(51) Int. Cl. A61B 5/1455

(52) U.S. Cl. CPC A61B 5/14552
USPC 600/340

(57) ABSTRACT

The disclosure relates to finger pulse oximetry sensors configurations including, for example, removable sensor sleeves, removable sensor pads, and light blocking configurations.

### CROSS-REFERENCE TO RELATED APPLICATIONS

This application claims the benefit of priority from U.S. Provisional Application No. 61/523,161, entitled Fingertip Pulse Oximeter, filed Aug. 12, 2011, which is incorporated in its entirety by reference herein.

### BACKGROUND OF THE INVENTION

Pulse oximetry is a widely accepted noninvasive procedure for measuring the oxygen saturation level of arterial blood, an indicator of a person's oxygen supply. A pulse oximetry system consists of a sensor applied to a patient, a monitor, and a patient cable connecting the sensor and the monitor. The sensor is attached to a tissue site, such as a patient's finger. The sensor has an emitter usually configured with both red and infrared LEDs that, for finger attachment, project light through the fingernail and into the blood vessels and capillaries underneath. Some optical based patient monitors have additional LEDs and can measure other physiological parameters. A detector is positioned at the fingertip opposite the fingernail so as to detect the LED emitted light as it emerges from the finger tissues. There are various noise sources for a sensor including electromagnetic interference (EMI), ambient light and piped light. Light that illuminates the detector without propagating through the tissue site, such as ambient light and piped light, is unwanted optical noise that corrupts the desired sensor signal. Ambient light is transmitted to the detector from external light sources, i.e. light sources other than the emitter. Piped light is stray light from the emitter that is transmitted around a tissue site along a light conductive surface, such as a reflective inner surface of face stock material, directly to the detector.

Pulse oximetry sensors can be relatively difficult to keep clean and properly sanitize because the internal areas are difficult to reach even with regular cleanings. Over time the sensors begin to build up grime in areas that are difficult to clean.

Further, when pulse oximeters are dropped, the fragile internal components can be broken or damaged by the impact. Many pulse oximeters have hard plastic housings that transfers the impact directly to the internal components. The impact can damage the components or connectors resulting in erroneous readings, ultimately, forcing medical practitioners to replace the malfunctioning or broken sensors.

### SUMMARY OF THE INVENTION

One aspect of the present disclosure includes a pulse oximetry sensor that advantageously provides EMI shielding and optical shielding, including multiple barriers to ambient light. In one embodiment a physiological sensor has a first shell housing including an emitter assembly, a second shell housing having a detector assembly, a first side wall and a second sidewall and a sensor chamber. The sensor chamber is a cavity between the first and second shell housings. The cavity is configured to engage a human finger between the first shell housing and the second shell housing. The first shell housing is coupled to the second shell housing such that it can be manipulated to increase the size of the tissue site to engage a portion of human tissue. The first and second side walls are configured to shield the sensor chamber from ambient light regardless of the position of the first housing and second shell housing relative to each other.

In another embodiment the sensor has a plastic sleeve having an interior side and an exterior side, wherein the interior side is configured to engage the human finger and the exterior side is configured to engage the sensor chamber.

In another embodiment the pulse oximeter also has a mounting dock. The mounting dock has a post and a base. The post is sized such that it fits within the sensor chamber and the post is configured to emit UV light within the sensor chamber.

Another aspect of the present disclosure provides a pulse oximeter with an emitter assembly that decouples from the sensor body. In one embodiment a physiological sensor includes a rigid housing having a top portion, a bottom portion and a sensor chamber. The top portion and the bottom portion are pivotally coupled together. The bottom portion has a detector assembly. The sensor chamber is a cavity between the top portion and the bottom portion of the housing. An emitter assembly is configured to engage the top portion. A flexible linkage is coupled to the emitter assembly and the bottom portion. The linkage is configured to elastically deform such that the emitter assembly can decouple from the top portion of the housing. The emitter assembly is configured to seal the top portion of the housing and shield the sensor chamber from ambient light when coupled to the housing.

In another embodiment the flexible linkage is an elastomeric material.

Another aspect of the present disclosure provides a cushioned sleeve for sanitation and comfort for the patient. In one embodiment a fingertip pulse oximeter includes a housing having an emitter assembly, a detector assembly, and a sensor chamber. The sensor includes a sleeve including, a top portion having a first hole and a bottom portion having a second hole. The sleeve is configured to be removably coupled within the sensor chamber. When the sleeve is coupled within the sensor chamber the first hole is configured to correspond to the position of the emitter assembly, and the second hole is configured to correspond to the position of the detector assembly. In some embodiments the sleeve is manufactured from a moldable foam.

Another aspect of the present disclosure provides shock resistant sensors. In one embodiment a physiological sensor has a sensor housing with a connector interface, and a connector housing surrounding the connector interface. The connector housing forms a cavity between the connector interface and an outer edge of the connector housing. A cable having a cable connector is coupled to the connector interface and the cable connector is at least partially recessed within the connector housing.

In another embodiment a physiological sensor has a sensor housing made from a rigid material. An internal frame having a plurality of mounting tabs is mounted to the sensor housing. The internal frame is made from a semi-rigid material. A mounting region, is configured to mount a printed circuit board to the plurality of mounting tabs. The frame is configured to absorb force transferred from the sensor housing such that only a portion of the force is transferred to the printed circuit board. In some embodiments the the plurality of mounting tabs are an elastomeric material

### BRIEF DESCRIPTION OF THE DRAWINGS

FIG. 1 illustrates a perspective view of an embodiment of a pulse oximetry sensor.

FIG. 2 illustrates a side view of the embodiment of the pulse oximetry sensor from FIG. 1.

FIG. 3 illustrates a side view of the embodiment of the pulse oximetry sensor from FIG. 1 with a finger inserted within the sensor.

FIG. 4 illustrates a back view of the embodiment of the pulse oximetry sensor from FIG. 1 with sensor in the open position.

FIG. 5 illustrates an embodiment of a finger sleeve.

FIG. 6 illustrates the embodiment of finger sleeve on a finger prior to being inserted into a fingertip pulse oximetry sensor.

FIG. 7 illustrates another embodiment of a pulse oximetry sensor in a first position.

FIG. 8 illustrates the embodiment of the pulse oximetry sensor from FIG. 7 in a second position.

FIG. 9 illustrates the embodiment of the pulse oximetry sensor from FIG. 7 in a third position.

FIG. 10 illustrates another embodiment of a finger sleeve.

FIG. 11 illustrates the embodiment of the finger sleeve from FIG. 10 inserted into an embodiment of a pulse oximetry sensor.

FIG. 12 illustrates an exploded view of the embodiment of the finger sleeve and pulse oximetry sensor from FIG. 11.

FIG. 13 illustrates an embodiment of a pulse oximetry sensor and an embodiment of a sensor dock.

FIG. 14 illustrates a plurality of sensor docks coupled together and a plurality of pulse oximetry sensors.

FIG. 15 illustrates an embodiment of a pulse oximetry sensor with a shock resistant connector housing.

FIG. 16 illustrates an exploded view of a schematic representation of an embodiment of a pulse oximetry sensor.

FIG. 17 illustrates an embodiment of an internal frame for a pulse oximetry sensor.

FIG. 18 illustrates another embodiment of an internal frame for a pulse oximetry sensor.

FIG. 19 illustrates a perspective view of another embodiment of a pulse oximetry sensor.

FIG. 20 illustrates a side view of the embodiment of the pulse oximetry sensor from FIG. 19.

FIG. 21 illustrates a side view of the embodiment of the pulse oximetry sensor from FIG. 19 with a finger inserted within the sensor.

FIG. 22 illustrates a back view of the embodiment of the pulse oximetry sensor from FIG. 19 with sensor in the open position.

FIG. 23 illustrates a perspective view of an embodiment of a pulse oximetry sensor.

FIG. 24 illustrates a side view of the embodiment of the pulse oximetry sensor from FIG. 23.

FIG. 25 illustrates a side view of the embodiment of the pulse oximetry sensor from FIG. 23 with a finger inserted within the sensor.

FIG. 26 illustrates a back view of the embodiment of the pulse oximetry sensor from FIG. 23 with sensor in the open position.

### DETAILED DESCRIPTION OF THE PREFERRED EMBODIMENT

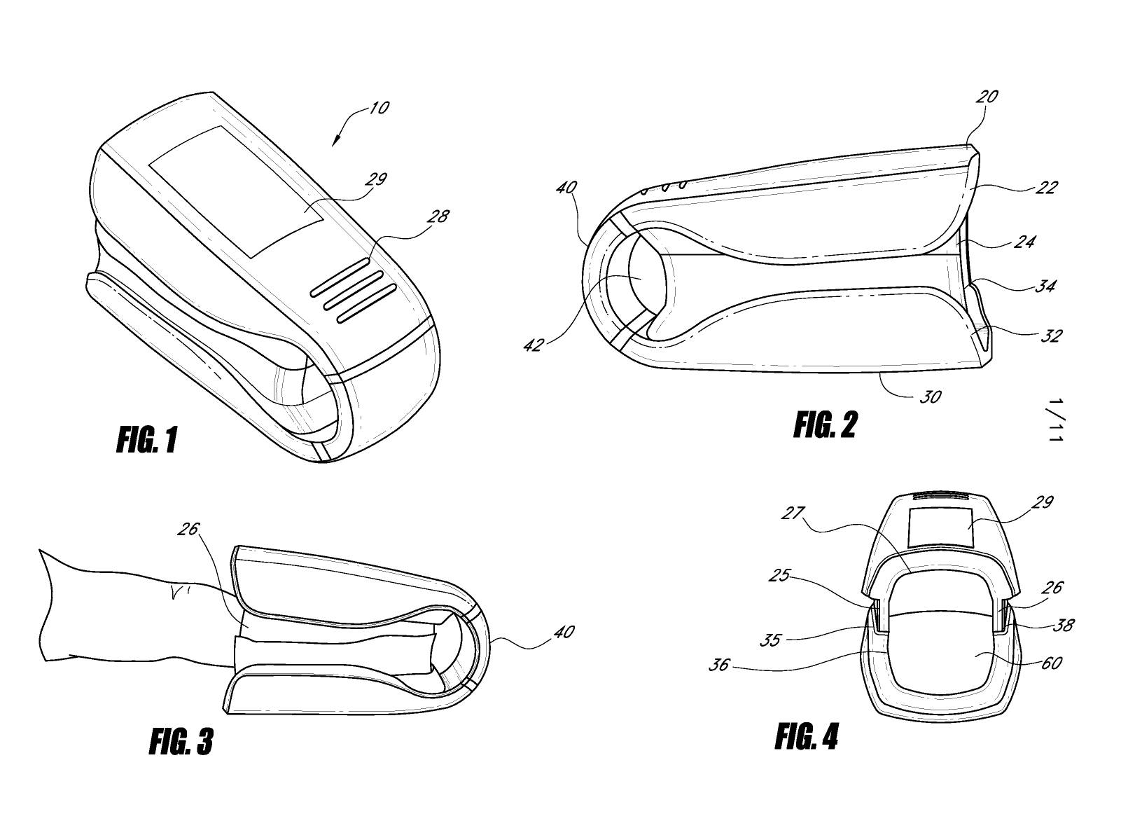

FIGS. 1 through 4 illustrate an embodiment of a fingertip pulse oximetry sensor 10. The sensor 10 is configured to communicate with a base station and is part of a physiological measurement system. A physiological measurement system allows the monitoring of a person, including a patient. In particular, the sensor 10 allows the measurement of blood constituent and related parameters in addition to oxygen saturation and pulse rate. The sensor 10 is adapted to attach to a tissue site, such as a fingertip. In this embodiment, the sensor 10 is incorporated into a reusable finger clip adapted to removably attach to, and transmit light through, a fingertip. In other embodiments, a sensor can be configured to attach to various tissue sites other than a finger, such as a foot or an ear. Also a sensor can be configured as a reflectance or transflectance device that attaches to a forehead or other tissue surface. The sensor 10 has onboard memory that allows it to record the signals monitored from the patient. The sensor 10 has sufficient storage capacity to store at least one night of data, but can store data for longer periods of time depending on the size of the memory and the sample rate of the data.

The sensor 10 includes a first, or upper, housing 20 that houses a multiple wavelength emitter assembly and a second, or lower, housing 30 that houses a corresponding detector assembly. The first and second housings 20, 30 form a tissue cavity 60. The tissue cavity has an emitter region on the upper housing 20 and a detector region on the lower housing 30. Alternatively, the emitter region can be in the lower housing 30 and the detector region can be in the upper housing 20.

An elastomeric region 40 is connected to the first housing 20 and the second housing 30. The elastomeric region forms an opening 42 between the housings and the elastomeric member. Preferably the opening 42 is configured such that a lanyard fits through the opening 42. The upper housing 20 and the lower housing 30 are pivotally coupled together within the housing. The coupling can be a spring or similar apparatus configured such that the upper housing 20 and lower housing 30 can expand and contract relative to each other in order to apply pressure to a fingertip inserted within the cavity 60. Preferably the pivot point of the sensor 10 is well behind the fingertip, which improves finger attachment and more evenly distributes pressure along the finger. One type of spring assembly is disclosed in U.S. Pat. No. 7,596,398, which is incorporated herein by reference in its entirety.

The first housing 20 further includes a first outer, or upper, shell 22, a first upper sidewall 24, a second upper sidewall 26, a upper sidewall edge 25, and an upper cavity wall 27. The first upper sidewall 24 extends substantially towards the second housing 30. The upper sidewall edge 25 is substantially perpendicular to the first upper sidewall 24. The second upper sidewall 26 is substantially parallel to the first upper sidewall 24. The upper cavity wall 27 defines the upper portion of the of the tissue cavity 60. In this embodiment the outer shell 22 further includes a plurality of textured regions 28 and an in mold laminate LCD screen 29. The LCD screen 29 is configured to display parameters that are measured by the sensor. For example the LCD screen 29 can be configured to display pulse rate and oxygen saturation. In some embodiments the upper or lower housing can have a USB connector interface.

The second housing 30 further includes a second outer, or lower, shell 32, a lower sidewall 34, a lower edge 35, a lower sidewall cavity 38, and a lower cavity wall 36. The lower sidewall 34 extends substantially toward the first housing 20. The lower edge 35 is substantially perpendicular to the lower sidewall 34. The lower cavity wall 36 defines the lower portion of the tissue cavity 60. The lower sidewall cavity is a cavity formed in the lower housing 30 between the lower sidewall 34 and the lower cavity wall 36. The lower cavity 38 is configured such that the second upper sidewall 26 can fit within the cavity 38. Preferably the cavity 38 is configured such that the second upper sidewall 26 can freely slide in and out of the cavity 38 when the first and second housings 20, 30 are manipulated about the pivot point.

When the sensor 10 is in the closed position, as illustrated in FIGS. 1 and 2, the upper sidewall edge 25 and the lower sidewall edge 35 are flush. The second upper sidewall 26 is fully enclosed within the lower cavity 38. Preferably the first upper sidewall 24 and the lower sidewall 34 have a substantially uniform surface.

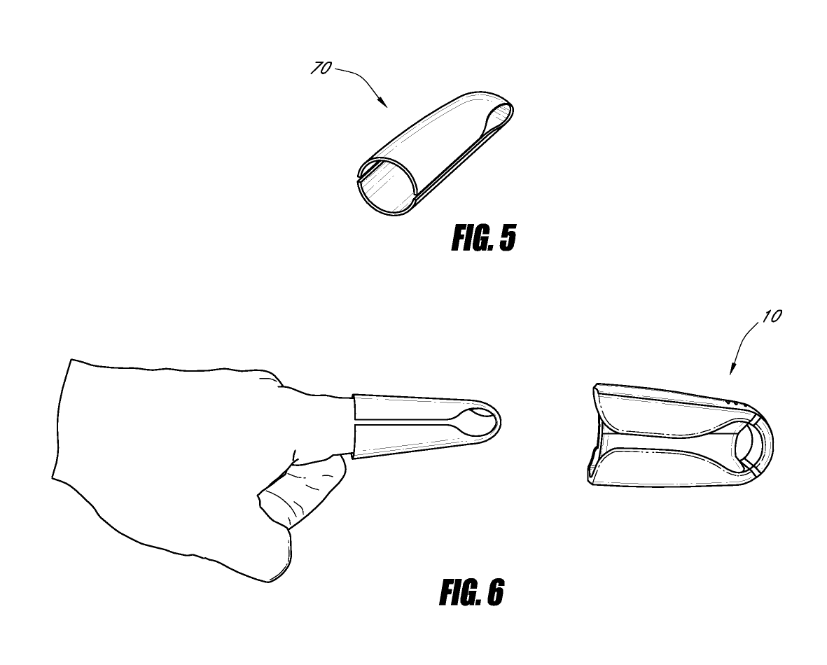

When the sensor 10 is in an open position, as illustrated in FIGS. 3 and 4, the upper sidewall edge 25 and lower sidewall edge 35 are separated from each other. The second upper sidewall 26 is at least partially exposed to the environment and ambient light. At least a portion of the second upper sidewall 26 is partially enclosed in the lower cavity 38. Preferably the second upper sidewall 26 is configured such that it is partially enclosed in the lower cavity regardless of the position of the first and second housings relative to one another. As such the tissue cavity remains closed off from the environment and ambient light regardless of the size of the fingertip inserted within the sensor. Preferably the second upper sidewall 26 is configured such that the sensor 10 such that the sensor 10 lets in the same amount of ambient light regardless of whether the sensor 10 is in an open or closed position.

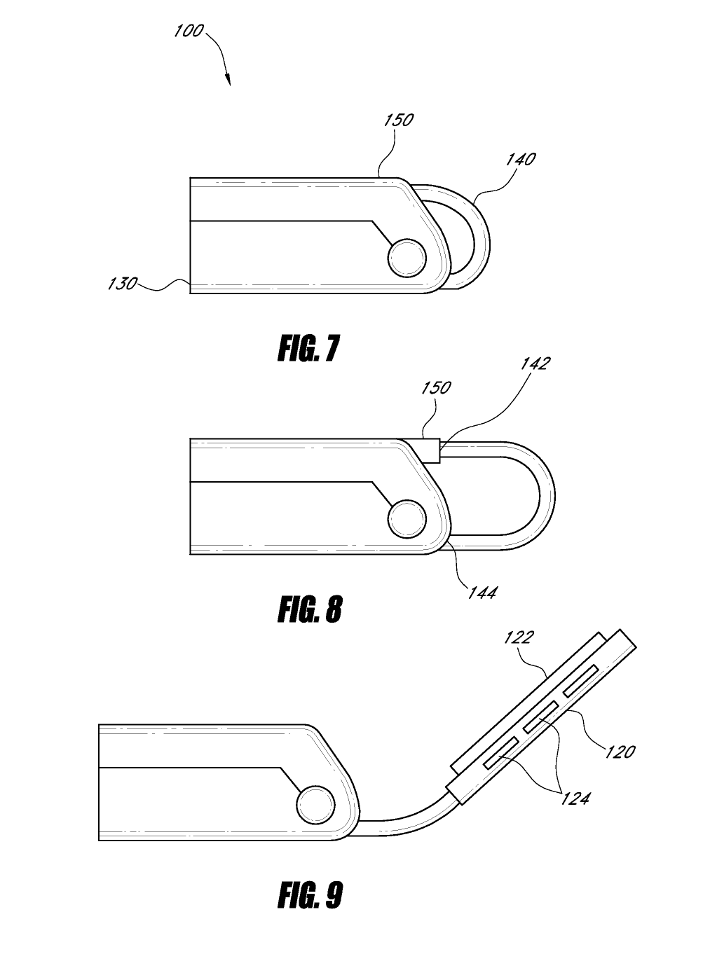

FIGS. 5 and 6 illustrate an embodiment of a disposable finger sleeve 70 for use with the sensor 10. The disposable finger sleeve 70 is a pre-formed finger cover formed of a transparent plastic material for use in a fingertip pulse oximetry sensor. Preferably, the sleeve 70 is a single size that can be universally used by patients regardless of the size of their fingers or the type of fingertip pulse oximetry sensor. Preferably the sleeve 70 is made of a transparent plastic material that does not disrupt or inhibit the operation of the sensor 10. Preferably the sleeve 70 is placed on the patient's finger prior to use of the sensor in order to keep the inner surfaces clean and sanitary. Alternatively the finger sleeve can be an opaque material with transparent window to allow operation of the sensor while providing additional shielding from ambient light and light piping. The sleeve can be made of resilient material, such as a hard or semi-hard plastic, such that the sleeve flexes open at the seams of the sleeve so as to conform to the finger, but generally retains its shape.

### Removable Emitter Assembly

FIGS. 7 through 9 illustrate another embodiment of a pulse oximetry sensor 100. In this embodiment the sensor has a first, or upper, housing 150, a second, or lower, housing 130, a frame 120, a tissue cavity (not shown), and a flexible linkage 140. The flexible linkage has a first end 142 and a second end 144. The first end 142 is coupled to first housing 120 and the second end 144 is coupled to the second housing 130. The flexible linkage 140 can be an elastomeric material. The second housing 130 further includes an emitter region or a detector region.

The first housing 120 further includes a sensor region 122 that can be an emitter or detector region. At least a portion of the first housing 120 is coupled to the flexible linkage 140. The first end 142 of the flexible linkage 140 is coupled to an end of the first housing 120. In some embodiments a portion of the first housing 120 can be encased in an elastomeric skin. The elastomeric skin can make up part of the flexible linkage 140. The first housing 120 is removably coupled to the frame 150. The first housing 120 includes a means for coupling the first housing 120 to the frame 150. In this embodiment the first housing 120 has a series of engaging members 124 that mate with similar engaging members on the frame 150. In other embodiments the first housing can use other means or methods of coupling the first housing 120 with frame 150.

The frame 150 is pivotally connected to the second housing 130. The frame 150 has a series of slots or grooves that allow the first housing 120 to couple with the frame 150. The frame has a plurality of coupling members (not shown) in order to facilitate the coupling and decoupling of the first housing 120 with the frame 150. In this embodiment the coupling member are a plurality of slots or grooves (not shown) that matches the engaging members 124 on the first housing 120.

Additionally FIGS. 7 through 9 illustrate a method for coupling the first housing 120 and the frame 150. Referring specifically to FIG. 7, the first housing 120 is illustrated in a first, or locked, position. In the first position the first housing 120 is coupled with the frame 150 and the coupling members are fully engaged with the engaging members 124. The pulse oximetry sensor is only operated when it is in the first position. Preferably, in the first position, the first housing 129 is coupled with the frame 150 such that the first housing 120 maintains its position during normal operation of the sensor 100. In some embodiments the first housing can be locked into position using a separate apparatus, such as a latch.

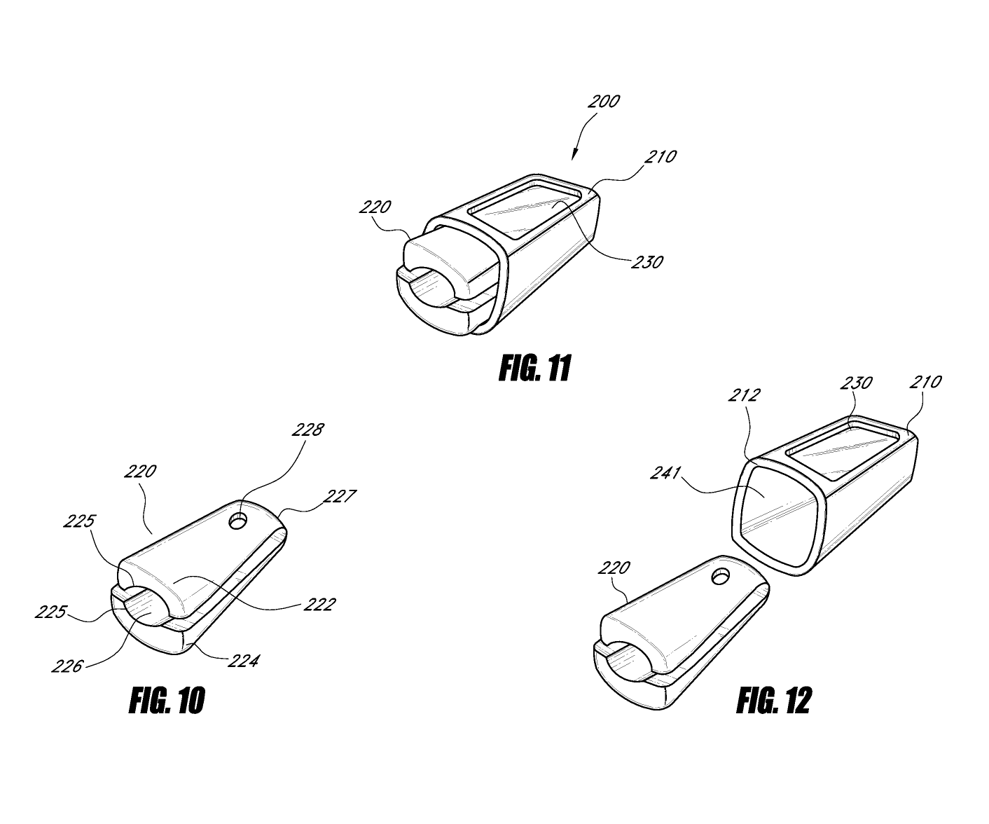

FIG. 8 illustrates when the first housing 120 is in transition from the first position to a second position. In the transitional position the first housing 120 is manipulated so that it is no longer locked in the first position. Preferably the first housing 120 can be removed from the frame 150 by sliding or manipulating the first housing such that the engaging members 124 are decoupled from the coupling members. The flexible linkage 140 is capable of elastic deformation such that the first housing 120 can be manipulated in order to decouple from the frame without disconnecting the flexible linkage 140 from the second housing 130.

FIG. 9 illustrates the sensor 100 in the second position where the first housing 120 has been decoupled from the frame 150. The first housing 120 remains coupled to the flexible linkage 140 and by extension to the second housing 130. In the second position the inside of the cavity is exposed. Preferably this allows access to the inside surfaces of the sensor 100, which would allow a practitioner to properly clean and sterilize the internal surfaces of the sensor 100, including the emitter region 122 and the detector region.

### Comfort Fit Finger Cushions

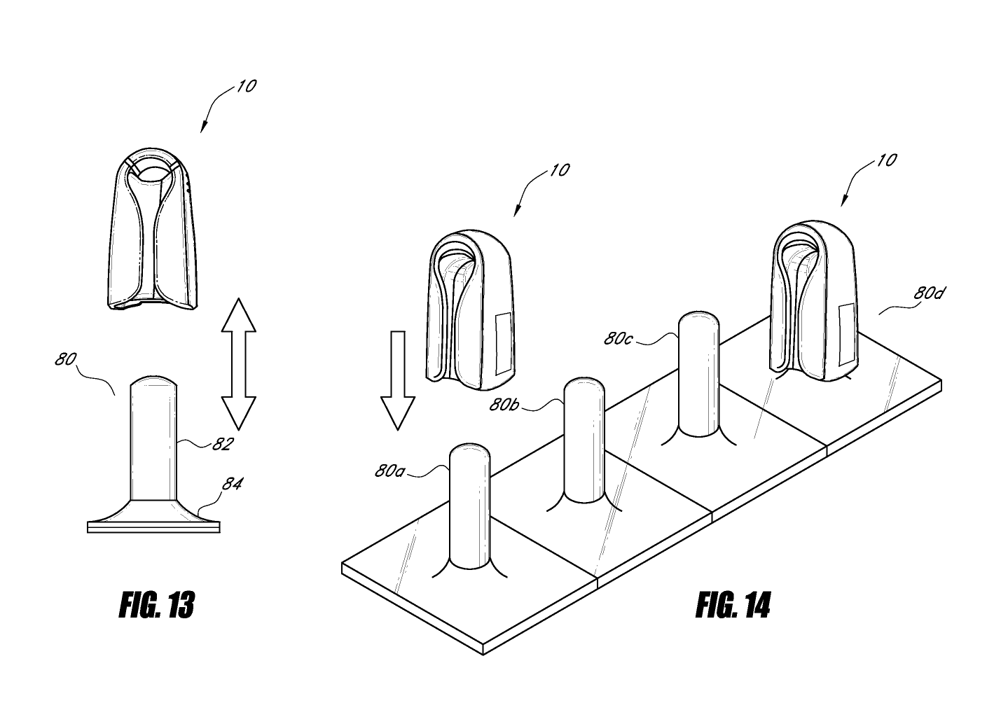

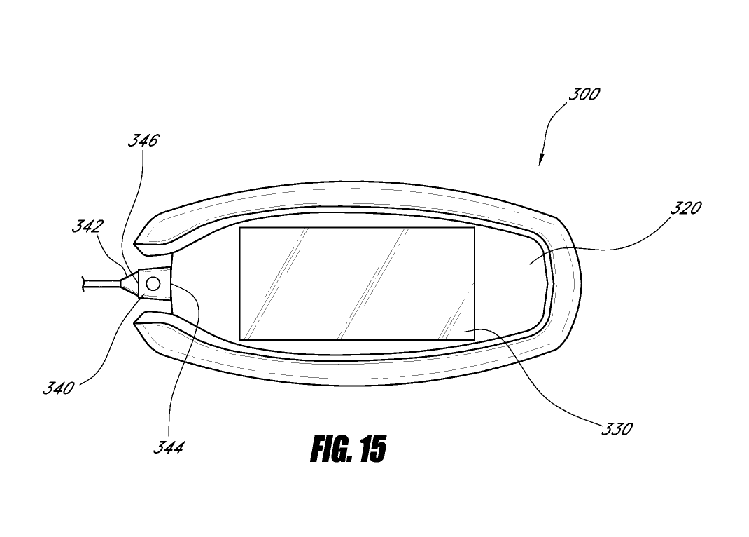

FIGS. 10 through 12 illustrate another embodiment of a fingertip pulse oximeter 200. In this embodiment the pulse oximeter sensor 200 has a housing 210, a sensor sleeve 220, and an LCD screen 230. The housing 210 has an outer wall 212 that defines an internal cavity 214. The internal cavity is configured to house the sensor sleeve 220. The housing has an emitter assembly and a detector assembly. Either the emitter assembly or detector assembly is on the upper side of the cavity 214, and the other assembly is on the lower side of the cavity 214. The LCD screen 230 is configured to display parameters that are measured by the sensor. For example the LCD screen 230 can be configured to display pulse rate and oxygen saturation.

The sensor sleeve 220 further includes a top portion 222, a bottom portion 224, and a fingertip region 226. The top portion 222 and the bottom portion 224 of the sleeve are connected at a distal end 227 and have a clam shell design that forms the fingertip region 226. Preferably the sleeve 220 is formed from a single piece of material. The top portion 224 and bottom portion 226 have molded regions 225 that are configured to accommodate a fingertip. The top portion 222 has a first hole 228. When the sleeve 220 is inserted into the housing 210, the first hole 228 is configured to align with the emitter assembly of the housing 210. The bottom portion also has a second hole (not shown) that is aligned with the detector assembly when the sleeve 220 is inserted in the housing 210. The first hole 228 and second hole allow light to pass through and are configured such that the emitter assembly of the sensor can properly transmit data to the detector assembly of the housing. In some embodiments the emitter assembly and detector assembly can be insert molded into the disposable cushion. Preferably, the sensor sleeve 220 is made from a soft pliable breathable material, such as foam. The sleeve material can be made of moldable foam that molds and contours to the patient's finger, thus providing a more comfortable or custom fit. Preferably the cushion sensor sleeves are disposed of after use and bacterial contamination between patients can be prevented. Different sleeves can be provided of different sizes that can be used to fit a wider range of finger sizes and shapes.

Preferably, the housing 210 is coupled to a patient cable, which transmits the data back to a physiological measurement system. Alternately, the housing 210 can include memory and/or wireless communication capability in order to store for later retrieval and/or wirelessly transmit data back to a physiological measurement system. The cable portion and housing 210 of the sensor stays clean and can be reused. Preferably the sleeve portion 220 is disposable. Alternatively the housing 210 can include wireless transmission radios to wirelessly transmit data.

### UV Light/Modular Charging Base Station

FIGS. 13 and 14 illustrate an embodiment of a pulse oximeter dock 80. The dock has a post 82 and a base 84. The post 82 has a light source for emitting UV light. The light source can emit light in all directions from the post 82. Preferably the dock 80 acts as charging station for the pulse oximetry sensor 10. The base 84 is configured such that it can be coupled with other bases 84, such that multiple docks 80 can be coupled together.

The UV light source can be used to clean the inside of the pulse oximeter 10. UV light can efficiently kill bacteria in areas that are difficult to clean and access. Preferably, dock 80 also serves as a charging station for the sensor 10. For example, the dock 80 can be configured to charge a lithium ion or other rechargeable battery. Preferably the post 82 is sized and shaped such that the sensor 10 can be easily coupled and decoupled from the post.

A plurality of docks 80 coupled together can be used to provide a convenient location for medical personnel to charge and check out pulse oximeters 10 for patients. The charging dock can also include such features as wireless connectivity to a base station. In situations where there is not a large amount of space to accommodate a full size patient monitor, the charging and connectivity portions of the dock can be split into two parts. One part consists of the main box which contains all of the processing components such as the PCBs, modules, batteries, etc. The other part is a simplified dock that can charge the handheld instrument and serves as a connectivity hub. Since the main box can be quite large, this portion can be placed away from the bedside area so that it does not take up essential real estate near the bedside. The smaller dock can be placed near the bedside. This serves as the main point of interface where a hand held device or tablet device resides. In addition the docking station can be mounted to using a mounting bracket, which can be attached in a convenient location near the bedside. The main box is the larger box and can be placed in a non-essential area of the room, which allows for more real estate near the bedside.

In some embodiments the dock portion can be transported with the patient cables. Generally the patient cable is wrapped around the unit itself or the cables are stuffed inside the handle opening, which can make it difficult to carry the instrument as well as manage the patient cable. Preferably the handle of the dock can extend outward, in a telescoping manner. The patient cords can be wrapped around the neck of the handle. The handle can be pushed in so that it is flush with the outer surface of the dock when it is not in use.

### Shock Resistant Connector Housing

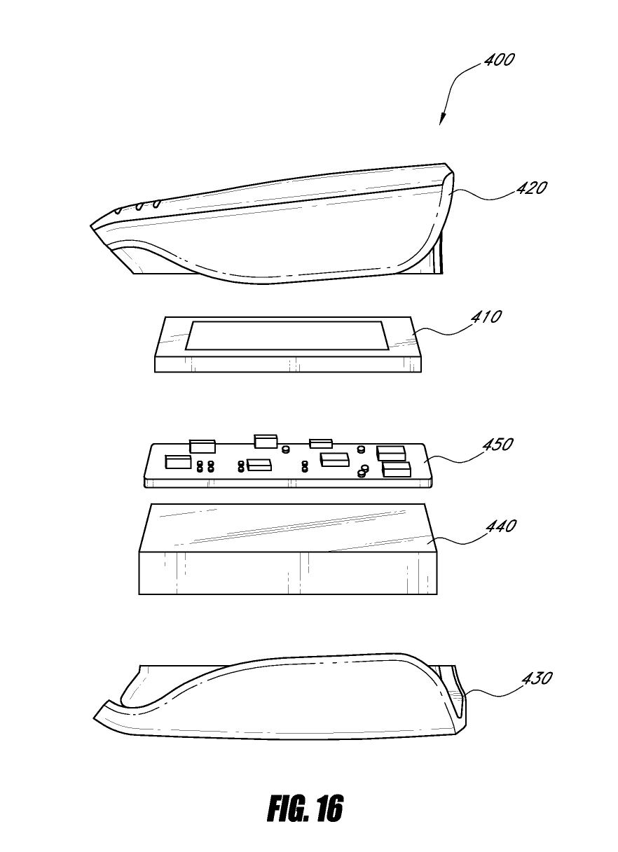

FIG. 15 illustrates another embodiment of a fingertip pulse oximetry sensor 300. In this embodiment the sensor includes a sensor housing 320, a connector housing 340, and an LCD screen 330. A connector interface 344 is configured to couple with a cable connector 342. The cable connector can be for a cable connected to a physiological measurement system, a power cable, a data cable, or any other cable used for the operation of a sensor. The connector housing 340 surrounds the connector interface 344, such that it creates a cavity between the connector interface 344 and the outer edge 346 of the housing 340. The connector interface 344 is recessed within the connector housing 340, such that the cable connector 342 is surrounded by the connector housing 340 when the cable is coupled to the sensor 300. Preferably there is a gap between the connector 342 and the connector housing 340 when the connector 342 is coupled to the sensor 300. The connector housing 340 can be constructed from a rigid or semi rigid material. In some embodiments the connector housing 340 can be constructed from an elastomeric material.

The connector housing 340 protects the cable connector 342 from possible damage to the connector in case the sensor is dropped or subject to an impact force. Generally, the sensor has a high probability that it may fall on the floor with the connector 342 inserted into the interface 344. Generally devices have a fully exposed connector which leaves them susceptible to breaking or damaging if the connector receives a hard impact. The connector housing 340 protects the connector 342 from damage caused by such impact. The connector housing 340 is configured to absorb the force of the impact and minimize the amount of force transferred to the connector 342, which helps prevent the connector 342 from receiving direct impact that can potentially damage the connector 342. In some embodiments the sensor housing may have an elastomeric sleeve or other material to help prevent damage to the sensor 300 and connector 342.

### Shock Resistant Sensors

FIG. 16 illustrates a simplified assembly of a pulse oximetry sensor 400. The assembly includes an first, or upper, housing 420, an LCD display 410, a printed circuit board (PCB) 450, a battery 440, and a second, or lower, housing 430. The sensor has delicate components such as the PCB 450, LCD 410, and battery 460.

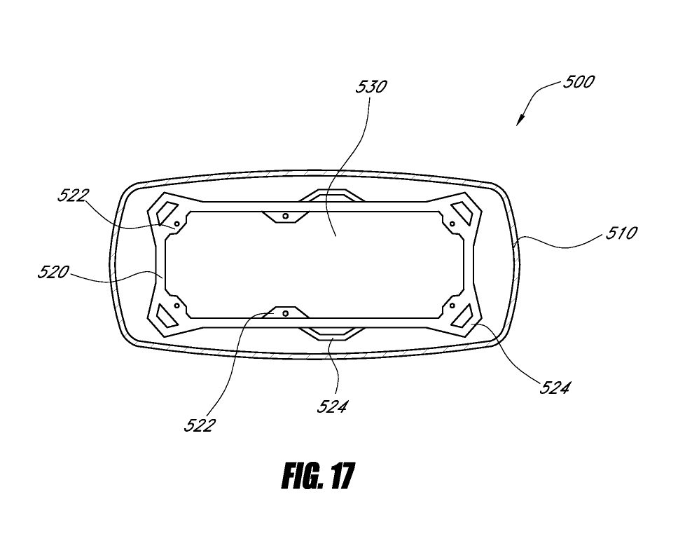

FIG. 17 illustrates a cross section of an embodiment of a housing of a pulse oximetry sensor 500. In this embodiment, the housing has a sensor housing 510, internal mounting frame 520, and a mounting region 530. The internal mounting frame 520 is a semi-rigid rubber structure that surrounds the mounting region 530. The internal mounting structure has a plurality of internal tabs or ribs 522 in a defined mounting pattern. Preferably the internal tabs 522 are made from the same material as the internal mounting structure 520. In some embodiments the internal tabs 522 can be made from a softer elastomeric material. The brackets 524 are for mounting the structure 520 to the sensor housing 510.

The delicate sensor components, such as the LCD and PCB can be mounted onto the internal tabs 522 in the mounting region 530. Generally each component would have different mounting patterns. In some embodiments, the frame can have a plurality of tabs configured to account for different mounting configurations. The soft pliable material of the frame 520 can potentially dampen any shock to the sensor 500. Preferably the frame 520 would be sandwiched between the top and bottom housing which are generally constructed of hard plastic. In some embodiments the top and bottom housing can have an elastomeric sleeve to further dampen the shock to the sensor.

FIG. 18 illustrates a cross section of another embodiment of a housing of a pulse oximetry sensor 600. In this embodiment, the housing includes a sensor housing 610 and an internal mounting frame 620. Preferably the sensor housing 610 is a semi-rigid or elastomeric material. The internal mounting frame 620 is a rigid structure with a plurality of internal tabs or ribs 622 in a defined mounting pattern. The internal tabs 622 are made from an elastomeric material. Preferably the internal tabs 622 are configured to align with the mounting pattern of the mounting component. The sensor housing 610 and the internal tabs 622 can potentially dampen any impact or shock to the delicate components mounted to the frame 620.

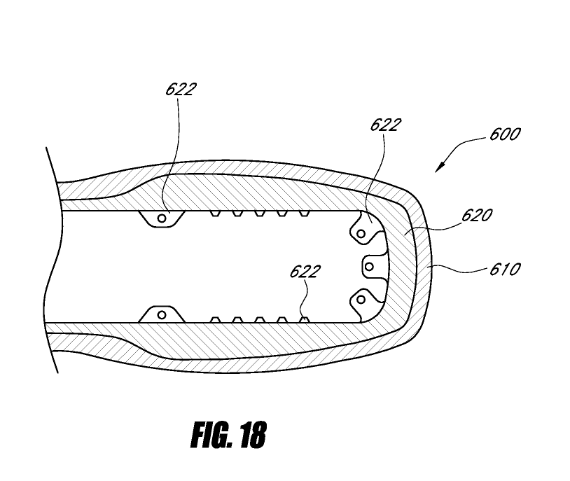

FIGS. 19 through 22 illustrate another embodiment of a pulse oximetry sensor 700. The sensor 700 has a first, or upper, housing 720 that houses a multiple wavelength emitter assembly and a second, or lower, housing 730 that houses a corresponding detector assembly. The first and second housings 720, 730 form a tissue cavity 760. The tissue cavity has an emitter region on the upper housing 720 and a detector region on the lower housing 730. Alternatively, the upper housing 720 has the detector region and the lower housing 730 has the emitter region. There is an opening 742 in the lower housing 730. The opening 742 can accommodate a lanyard. The upper housing 720 and the lower housing 730 are pivotally coupled together. The coupling can be a spring or similar apparatus configured such that the upper housing 720 and lower housing 730 can expand and contract relative to each other in order to apply pressure to a fingertip inserted within the cavity 760.

The first housing 720 has an upper region 727 that partially defines the cavity 760. The upper region 727 can be contoured to match the shape of a finger, in order provide a more comfortable and snug fit. The upper region 727 can have an elastomeric coating. In this embodiment the first housing 720 further includes a plurality of textured regions 728 and an in mold laminate LCD screen 729. The LCD screen 729 is configured to display parameters that are measured by the sensor. For example the LCD screen 729 can be configured to display pulse rate and oxygen saturation. In some embodiments the upper or lower housing can have a USB connector interface.

The second housing 730 has a lower region 736 and an outer, or lower, shell 732. The lower region 736 partially defines the cavity 760 and can be contoured to match the shape of a finger, in order to provide a more comfortable and snug fit. The lower region 736 can have an elastomeric coating. The outer shell encompasses the second housing 730. The outer housing has sidewalls 734a,b that extend toward the first housing 720 such that the first housing fits between the sidewalls 734. The sidewalls 734 define a portion of the cavity 760. The sidewalls are configured to extend substantially beyond the upper portion 727 of the first housing 720 and allow the first housing freedom to be manipulated relative to the sidewalls 734. The upper housing 720 and lower housing 730 have a plurality of textured grip regions 728. A user can squeeze the textured grips 728 together to open the sensor 700 and allow for finger placement.

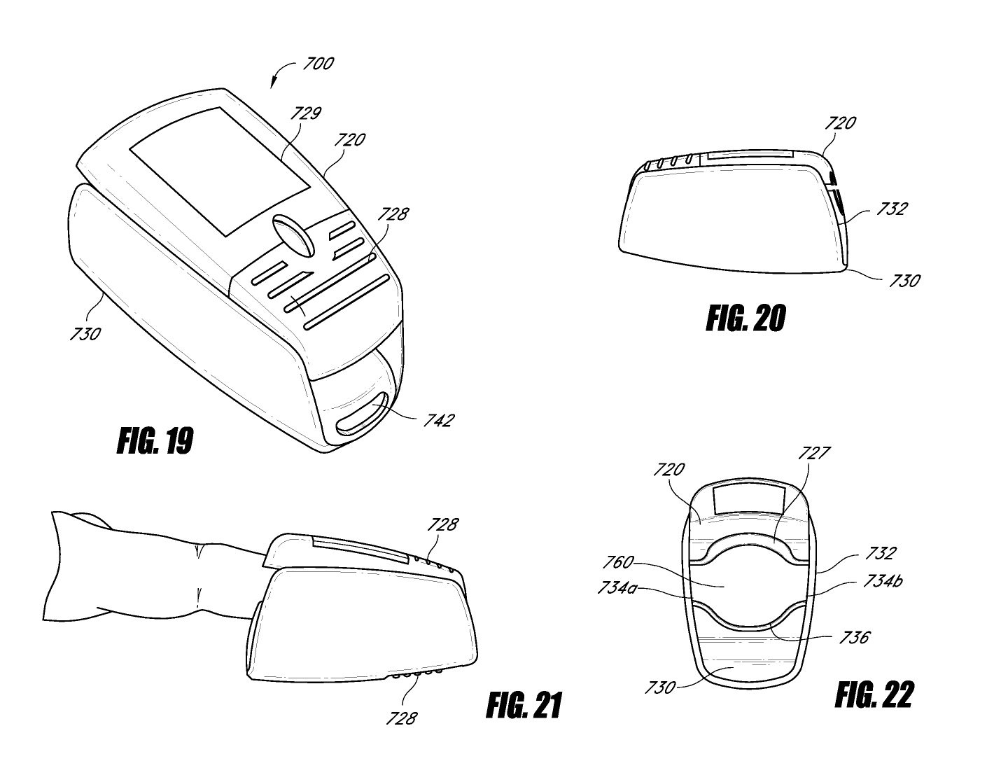

FIGS. 19 and 20 illustrate the sensor 700 in a closed position. FIGS. 21 and 22 illustrate the sensor 700 in an open position. The outer shell 732 is configured such that substantially the same amount of ambient light enters the tissue cavity in an open or closed position.

FIGS. 23 through 26 illustrate yet another embodiment of a pulse oximetry sensor 800. The sensor 800 has a first, or upper, housing 820 that houses a multiple wavelength emitter assembly and a second, or lower, housing 830 that houses a corresponding detector assembly. The first and second housings 820, 830 form a tissue cavity 860. The tissue cavity has an emitter region on the upper housing 820 and a detector region on the lower housing 830. Alternatively, the upper housing 720 has the detector region and the lower housing 730 has the emitter region. A loop region 840 forms an opening 842 on the sensor 800. Preferably the opening 842 is configured such that a lanyard fits through the opening 842. The upper housing 820 and the lower housing 830 are pivotally coupled together within the housings.

The first housing 820 further includes a first outer, or upper, shell 822, a first upper sidewall 824, a second upper sidewall 826, an upper sidewall edge 825, and an upper cavity wall 827. The first upper sidewall 824 extends substantially towards the second housing 830. The upper sidewall edge 825 is substantially perpendicular to the first upper sidewall 824. The second upper sidewall 826 is substantially parallel to the first upper sidewall 824. The upper cavity wall 827 defines the upper portion of the of the tissue cavity 860. In this embodiment the outer shell 822 further includes a plurality of textured regions 828 and an in mold laminate LCD screen 829. The LCD screen 829 is configured to display parameters that are measured by the sensor. For example the LCD screen 829 can be configured to display pulse rate and oxygen saturation. In some embodiments the upper or lower housing can have a USB connector interface.

The second housing 830 further includes a second outer, or lower, shell 832, a lower sidewall 834, a lower edge 835, a lower sidewall cavity 838, and a lower cavity wall 836. The lower sidewall 834 extends substantially toward the first housing 820. The lower edge 835 is substantially perpendicular to the lower sidewall 834. The lower cavity wall 836 defines the lower portion of the tissue cavity 860. The lower sidewall cavity is a cavity formed in the lower housing 830 between the lower sidewall 834 and the lower cavity wall 836. The lower cavity 838 is configured such that the second upper sidewall 826 can fit within the cavity 838. Preferably the cavity 838 is configured such that the second upper sidewall 826 can freely slide in and out of the cavity 838 when the first and second housings 820, 830 are manipulated about the pivot point. The cavity can be configured to enclose the inner and outer surfaces of the upper sidewall. The cavity can also be configured so that the outer surface of the upper sidewall is in the cavity and the inner surface of the upper sidewall is exposed to the tissue cavity. The upper housing 820 and lower housing 830 have a plurality of textured grip regions 828. A user can squeeze the textured grips 828 together to open the sensor 800 and allow for finger placement.

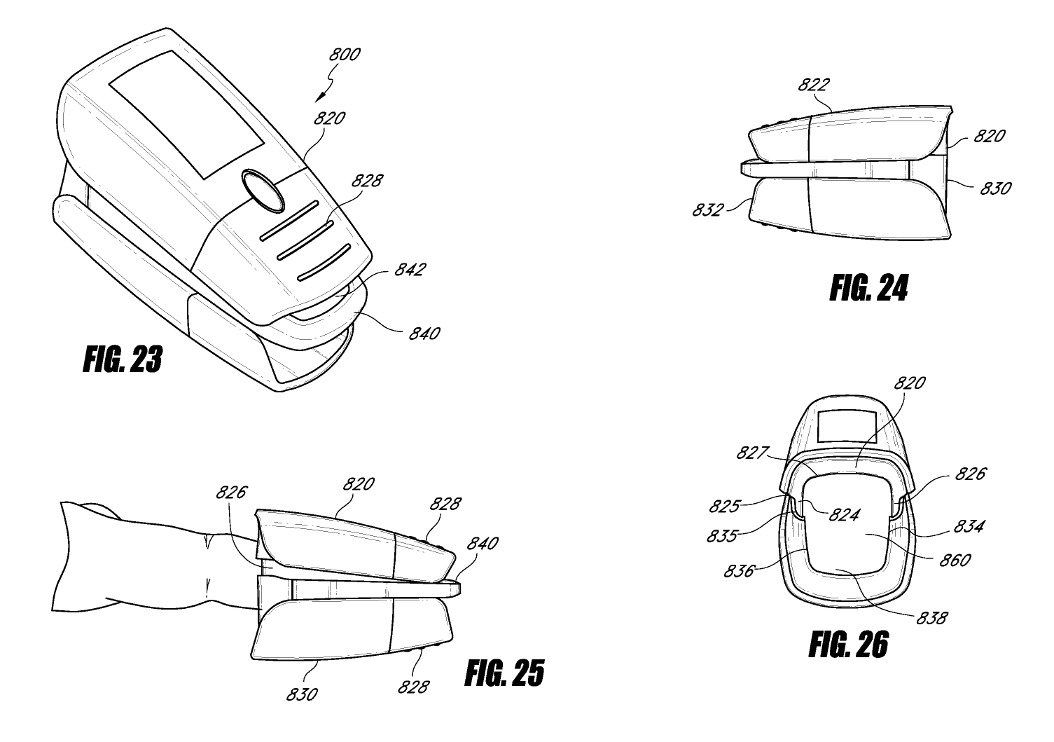

When the sensor 800 is in the closed position, as illustrated in FIGS. 23 and 24, the upper sidewall edge 825 and the lower sidewall edge 835 are flush. The second upper sidewall 826 is enclosed within the lower cavity 838. Preferably the first upper sidewall 824 and the lower sidewall 834 have a substantially uniform surface.

When the sensor 800 is in an open position, as illustrated in FIGS. 23 and 24, the upper sidewall edge 825 and lower sidewall edge 835 are separated from each other. The outer surface of the second upper sidewall 826 is at least partially exposed to the environment and ambient light. At least a portion of the second upper sidewall 826 is partially enclosed in the lower cavity 838. Preferably the second upper sidewall 826 is configured such that it is partially enclosed in the lower cavity regardless of the position of the first and second housings relative to one another. As such the tissue cavity remains closed off from the environment and ambient light regardless of the size of the fingertip inserted within the sensor. Preferably the second upper sidewall 826 is configured such that substantially the same amount of ambient light enters the tissue cavity in an open or closed position.

Although certain embodiments, features, and examples have been described herein, it will be understood by those skilled in the art that many aspects of the methods and devices illustrated and described in the present disclosure can be differently combined and/or modified to form still further embodiments. For example, any one feature of the physiological measurement system described above can be used alone or with other components without departing from the spirit of the present invention. Additionally, it will be recognized that the methods described herein may be practiced in different sequences, and/or with additional devices as desired. Such alternative embodiments and/or uses of the methods and devices described above and obvious modifications and equivalents thereof are intended to be included within the scope of the present invention. Thus, it is intended that the scope of the present invention should not be limited by the particular embodiments described above, but should be determined only by a fair reading of the claims that follow.

What is claimed is:

1. A physiological sensor comprising:
a first shell housing including one of an emitter or detector assembly;
a second shell housing including the other of the emitter or detector assembly, the first shell housing including a first side wall and a second sidewall;
a sensor chamber, wherein the sensor chamber is a cavity between the first and second shell housings, wherein the cavity is configured to engage a human finger between the first shell housing and the second shell housing;
the first shell housing is coupled to the second shell housing such that the first and second housings can be manipulated to increase the size of the sensor chamber to engage a portion of human tissue; and
the first and second side walls are configured to shield the sensor chamber from ambient light regardless of the position of the first housing and second shell housing relative to each other.

2. The sensor of claim 1 further comprising a plastic sleeve having an interior side and an exterior side, wherein the interior side is configured to engage the human finger and the exterior side is configured to engage the sensor chamber.

3. The sensor from claim 1 wherein the pulse oximeter further comprises:
a mounting dock, a post, and a base, wherein the post is sized such that it fits within the sensor chamber, the post is configured to emit UV light within the sensor chamber.

4. The sensor of claim 1, wherein the sensor further comprises a processor and a display, the processor configured to determine a physiological measurement based on detected sensor signals and the display configured to display the physiological measurement.

5. The sensor of claim 4, wherein the sensor is a stand alone pulse oximetry monitor.

6. The sensor of claim 5, wherein the sensor further comprises a transceiver configured to transmit measured physiological information.

7. A physiological sensor comprising:
a rigid housing having a top portion and a bottom portion, wherein the top portion and the bottom portion are pivotally coupled together, the bottom portion including one of an emitter or detector assembly;
a sensor chamber, having a cavity formed between the top portion and the bottom portion of the housing;
an upper housing having the other of the emitter or detector assembly configured to engage the top portion;
a flexible linkage coupled to the upper housing and the bottom portion;
wherein the linkage is configured to elastically deform such that the upper housing can decouple from the top portion of the housing; and
wherein the emitter assembly is configured to coupled to the top portion of the housing and shield the sensor from ambient light when coupled to the housing.

8. The sensor of claim 7 further comprising a plastic sleeve having an interior side and an exterior side, wherein the interior side is configured to engage the human finger and the exterior side is configured to engage the sensor chamber.

9. The sensor from claim 7 wherein the pulse oximeter further comprises:
a mounting dock, a post, and a base, wherein the post is sized such that it fits within the sensor chamber, the post is configured to emit UV light within the sensor chamber.

10. The sensor of claim 7, wherein the sensor further comprises a processor and a display, the processor configured to determine a physiological measurement based on detected sensor signals and the display configured to display the physiological measurement.

11. The sensor of claim 10, wherein the sensor is a stand alone pulse oximetry monitor.

12. The sensor of claim 11, wherein the sensor further comprises a transceiver configured to transmit measured physiological information.

13. A fingertip pulse oximeter comprising:
a housing having an emitter assembly, a detector assembly, and a sensor chamber;
a sleeve further comprising, a top portion having a first hole and a bottom portion having a second hole, wherein the sleeve is configured to be removably coupled within the sensor chamber; and
wherein when the sleeve is coupled within the sensor chamber the first hole is configured to correspond to the position of the emitter assembly, and the second hole is configured to correspond to the position of the detector assembly.

14. The fingertip pulse oximeter from claim 13 wherein the sleeve is manufactured from a moldable foam.

15. The fingertip pulse oximeter from claim 13, wherein the sleeve is resilient.

16. The fingertip pulse oximeter from claim 15, wherein the sleeve is plastic.

17. A physiological sensor comprising:
a sensor housing having a connector interface;
a connector housing surrounding the connector interface, wherein there is a cavity formed between the connector interface and an outer edge of the connector housing;
a cable having a cable connector, wherein the cable connector is coupled to the connector interface; and
wherein the cable connector is at least partially recessed within the connector housing.

18. A physiological sensor comprising:
a sensor housing, wherein the sensor housing is a rigid material;
an internal frame having a plurality of mounting tabs, wherein the internal frame is a semi-rigid material;
a mounting region, wherein the mounting region is configured to mount a printed circuit board to the plurality of mounting tabs; and
wherein the frame is configured to absorb force transferred from the sensor housing such that only a portion of the force is transferred to the printed circuit board.

19. The physiological sensor from claim 18 wherein the plurality of mounting tabs are an elastomeric material
